(19) United States
(12) Patent Application Publication
(10) Pub. No.: US 2026/0137962 A1
(43) Pub. Date: May 21, 2026

(54) CLOSED-LOOP TRANSCRANIAL ULTRASOUND BRAIN STIMULATION DEVICE

(71) Applicants: Affiliated Kangning Hospital of Ningbo University, Ningbo (CN); Tianjin Anding Hospital, Tianjin (CN)

(72) Inventors: Dongsheng ZHOU, Ningbo (CN); Shen LI, Tianjin (CN); Chang YU, Ningbo (CN); Xingxing LI, Ningbo (CN)

(21) Appl. No.: 19/010,198

(22) Filed: Jan. 6, 2025

(30) Foreign Application Priority Data
Nov. 20, 2024 (CN) 202411665830.6

(51) Int. Cl. A61N 7/00

(52) U.S. Cl. CPC A61N 7/00; A61N 2007/0026

(57) ABSTRACT

The disclosure relates to the field of medical equipment, and discloses a closed-loop transcranial ultrasound brain stimulation device, the device includes a head ring, where the outer wall of the head ring is fixedly connected with a vertically symmetrical connecting belt, the outer wall of the head ring is fixedly connected with a protective shell, the inside of the protective shell is rotatably connected with a knob I, the outer wall of the knob I is fixedly connected with a gear, the lower surface of the connecting belt is fixedly connected with a rack, and the rack meshes with the gear; the upper surface of the head ring is fixedly connected with a connecting rod, the upper surface of the connecting rod is fixedly connected with a fixed ring, the outer wall of the fixed ring is fixedly connected with a toothed ring.

### TECHNICAL FIELD

The disclosure relates to the technical field of medical equipment, and in particular to a closed-loop transcranial ultrasound brain stimulation device.

### BACKGROUND

Neurological disorders include central and peripheral nervous system disorders manifesting as sensory, cognitive, motor and other neurological dysfunctions, causing epilepsy, stroke, Parkinson's, Alzheimer's, migraine, etc. According to the data released by China Center for Disease Control and Prevention in May 2012, nervous system disorders rank among the top ten in the disease death list. Nervous system disorders have a serious impact on the quality of life of patients and their families, as well as being a major economic burden on families and society. In the treatment of nervous system disorders, transcranial ultrasound brain stimulation devices are necessary to assist physicians.

A multifunctional wearable ultrasonic therapeutic apparatus is disclosed in the application with the publication number of CN112972913A, which includes an ultrasonic transmitter, an ultrasonic connecting device, straps and a mounter mechanism, wherein the straps are symmetrically arranged on the ultrasonic transmitter, the lower end of the ultrasonic transmitter is provided with a connecting part, the ultrasonic connecting device is connected with the connecting part, and the mounter mechanism is matched with the ultrasonic connecting device; the back end of the ultrasonic transmitter is provided with a power preset mode regulator, one side of the power preset mode regulator on the ultrasonic transmitter is provided with a time preset mode regulator, the lower end of the power preset mode regulator on the ultrasonic transmitter is provided with a mode regulator, one side of the power preset mode regulator on the ultrasonic transmitter is provided with a temperature sensor, the upper end of the temperature sensor on the ultrasonic transmitter is provided with a loudspeaker, and the lower end of the temperature sensor on the ultrasonic transmitter is provided with an indicator light; the ultrasonic connecting device includes a connecting head, a converter, a connecting wire and an ultrasonic emitting probe, one end of the connecting head is connected with the connecting part, and the other end of the connecting head is connected with the converter through a wire. In the application with the publication number of CN112972913A, the ultrasonic generator is fixed by the patch design, causing instability of the ultrasound generator fixation and affecting the subsequent treatment efficacy. Meanwhile, it is difficult to adjust the position of the ultrasound generator, and it is not convenient to quickly adjust the position of the ultrasound generator according to specific conditions.

### SUMMARY

In view of the shortcomings of the prior art, the disclosure provides a closed-loop transcranial ultrasound brain stimulation device, so as to solve the problems of unstable fixation of the ultrasonic generator caused by fixing the ultrasonic generator through the patch design and the subsequent adverse effects on the treatment effect of patients.

In order to achieve the above objectives, the closed-loop transcranial ultrasound brain stimulation device includes a head ring, where the outer wall of the head ring is fixedly connected with a vertically symmetrical connecting belt, the outer wall of the head ring is fixedly connected with a protective shell, the inside of the protective shell is rotatably connected with a knob I, the outer wall of the knob I is fixedly connected with a gear, the lower surface of the connecting belt is fixedly connected with a rack, and the rack meshes with the gear; the upper surface of the head ring is fixedly connected with a connecting rod, the upper surface of the connecting rod is fixedly connected with a fixed ring, the outer wall of the fixed ring is fixedly connected with a toothed ring, the outer wall of the fixed ring is slidably connected with a moving block I, the inner part of the moving block I is slidably connected with a knob II, the outer wall of the knob II is fixedly connected with a connecting block, the inner part of the connecting block is rotatably connected with a pawl, the pawl is meshed with the toothed ring; the outer wall of the knob II is sleeved with a spring, one end of the spring is fixedly connected to the inside of the connecting block, the other end of the spring is fixedly connected to the inner wall of the moving block I; the outer wall of the moving block I is fixedly connected to a moving block II, the outer wall of the moving block II is slidably connected to the inside of the fixed ring, the outer wall of the moving block II is fixedly connected with an ultrasonic generator, and the upper surface of the head ring is provided with a fixed component.

Optionally, the fixed component includes a fixed plate, the lower surface of the fixed plate is fixedly connected to the upper surface of the head ring, a screw is screwed in the fixed plate, one end of the screw is fixedly connected with a handle II, and the other end of the screw is fixedly connected with a rubber pad.

Optionally, the inside of the ultrasonic generator is fixedly connected with a generator body, the inside of the ultrasonic generator is fixedly connected with a dustproof net I, the inside of the ultrasonic generator is rotatably connected with a rotating rod, the inside of the rotating rod is fixedly connected with an acoustic lens, and the outer wall of the acoustic lens is arranged inside the ultrasonic generator.

Optionally, one end of the rotating rod is fixedly connected with a handle I, the other end of the rotating rod is fixedly connected with a turntable, a limit rod is slidably connected inside the turntable, and the outer wall of the limit rod is slidably connected inside the ultrasonic generator.

Optionally, the outer wall of the head ring is fixedly connected with a temperature sensor, the upper surface of the fixed ring is fixedly connected with a centrally symmetric elastic band, the outer wall of the elastic band is fixedly connected with a coil, the bottom end of the elastic band is fixedly connected with a top plate, the upper surface of the top plate is fixedly connected with a biofeedback sensor, and the lower end of the outer wall of the elastic band is fixedly connected with a water bag.

Optionally, the outer wall of the water bag is fixedly connected with a magnet II, the outer wall of the magnet II is provided with a magnet I, the outer wall of the magnet I is fixedly connected with a shell, the inner wall of the shell is fixedly connected with a dustproof net II, the inside of the shell is fixedly connected with a fan, the inside of the shell is fixedly connected with a mounting plate, and the inside of the mounting plate is fixedly connected with a semiconductor refrigeration sheet, the refrigeration surface of the semiconductor refrigeration sheet is attached to the outer wall of the fan, and the heating surface of the semiconductor refrigeration sheet is fixedly connected with a radiating fin, and one end of the radiating fin is arranged on the outer wall of the shell.

Optionally, the inside of the head ring is rotatably connected with a connector I, the outer wall of the connector I is fixedly connected with an adjusting rod I, the inside of the adjusting rod I is rotatably connected with an adjusting handle, and the outer wall of the adjusting rod I is rotatably connected with an adjusting rod II.

Optionally, the bottom end of the adjusting rod II is fixedly connected with a connector II, the outer wall of the connector II is rotatably connected with a supporting block, and the outer wall of the supporting block is fixedly connected with a base.

Optionally, the inner upper end of the base is connected with a storage box in a sliding way, the upper surface of the base is fixedly connected with a display screen, the upper surface of the base is fixedly connected with a controller, the controller is electrically connected with an indicator light, the outer wall of the indicator light is fixedly connected with the upper surface of the base, and the controller is electrically connected with the temperature sensor.

Optionally, the inside of the base is fixedly connected with a processor, a collector is arranged below the processor, the outer wall of the collector is fixedly connected with the inside of the base, and the collector and the processor are electrically connected.

Working principle: when the ultrasonic brain stimulation device needs to be used, firstly, the patient is seated in the seat, and the adjusting rod II is pulled to rotate with the connector II in the supporting block, then the adjusting rod I with the connector 1 is rotated inside the head ring, the head ring is moved to the top of the patient's head, and the head ring is attached to the patient's head, the knob I is rotated to rotate with the gear in the protective shell, and the gear moves with the rack and the connecting belt inside the protective shell to adjust the closed loop of the head ring, so that the head ring is tightly attached to the patient's head; then, the handle II is rotated inside the fixed plate with the screw, and the rubber pad is attached to the patient's head, so that the head ring is attached to the patient's head more closely; then, the patient's brain waves collected by the biofeedback sensor on the top plate of the elastic band are collected by the collector connected with the base, processed by the processor, and finally displayed by the display screen, so that doctors can observe the patient's brain waves conveniently; when the position of the ultrasonic sensor needs to be moved, the moving block I is pulled, and the moving block I moves with the pawl on the outer wall of the fixed ring under the action of the toothed ring, so that the specific position of the ultrasonic sensor can be adjusted according to the specific situation of the patient; when it is necessary to change the ultrasonic characteristics reflected by the ultrasonic sensor, the rotating handle rotates with the rotating rod, and the rotating rod drives the acoustic lens to rotate inside the ultrasonic sensor, when the acoustic lens moves to a proper position, the limit rod is inserted into the ultrasonic sensor through the turntable, so that the position of the acoustic lens is fixed, which is convenient to change the position of ultrasonic gathering; the patient can be treated with ultrasonic stimulation through the ultrasonic sensor, and also be treated with magnetic stimulation through the electromagnetic wave generated by the coil connected with the elastic band, a variety of options are provided. When it is necessary to cool the patient's, firstly, the head of the patient is cooled by the water bag connected with the elastic band to prevent the brain from overheating, and when the temperature of the water bag or the patient's head rises, the head ring is connected with the temperature sensor to transmit information to the controller, and the controller controls the indicator light to light up to remind the doctor; when the patient's head need to be rapidly cooled, the shell is connected with the magnet I on the outer wall of the shell with the magnet II, the fan inside the shell is started and the semiconductor refrigeration sheet attached to the out wall of the fan is used for cooling the air inside the shell, and then the fan blows out the cooled air to rapidly cool the cooling water inside the water bag, so that the use experience of patients is improved. The heat generated by the heating surface of the semiconductor refrigeration sheet is discharged from the inside of the shell through the radiating fins connected with the heating surface of the semiconductor refrigeration sheet.

The closed-loop transcranial ultrasound brain stimulation device provided by the disclosure has the following beneficial effects:

First, the disclosure achieves the function of adjusting the size of the closed loop of the head ring through the cooperation between the connecting belt, the knob I, the protective shell, the gear and the rack, and solves the problem that the ultrasonic generator is fixed by the patch design, which is unstable in fixing the ultrasonic generator and affects the subsequent treatment effect on patients. Through the cooperation between the moving block I, the pawl, the connecting block, the knob II, the spring I, the moving block II and the ultrasonic generator, the function of quickly adjusting the position of the ultrasonic generator is achieved, and at the same time, the problem that the position adjustment of the ultrasonic generator is troublesome and inconvenient to adjust according to specific conditions is solved, and the functionality of the ultrasonic brain stimulation device is improved.

Second, through the cooperation of the generator body, the acoustic lens, the dustproof net I, the rotating rod, the handle I, the turntable and the limit rod, the disclosure achieves the function of conveniently adjusting the angle of the acoustic lens, changing the ultrasonic characteristics, and improving the application range of the device for treating patients; through the cooperation of the temperature sensor, the controller and the indicator light, it achieves the function of conveniently preventing brain overheating, and ensures the use safety of the device.

Third, through the cooperation of the water bag, the shell, the dustproof net II, the fan, the mounting plate, the semiconductor refrigeration sheet and the radiating fin, the water bag can be continuously cooled, and the effect of cooling the brain can be improved; through the cooperation of the water bag, the shell, the magnet I and the magnet II, the fan can be disassembled conveniently, so as to prevent the lower temperature of the water bag from affecting the user's experience and ensure the safety.

### BRIEF DESCRIPTION OF THE DRAWINGS

FIG. 1 is a perspective view of the present disclosure.

FIG. 2 is a schematic diagram of the two-part structure of the adjusting rod of the present disclosure.

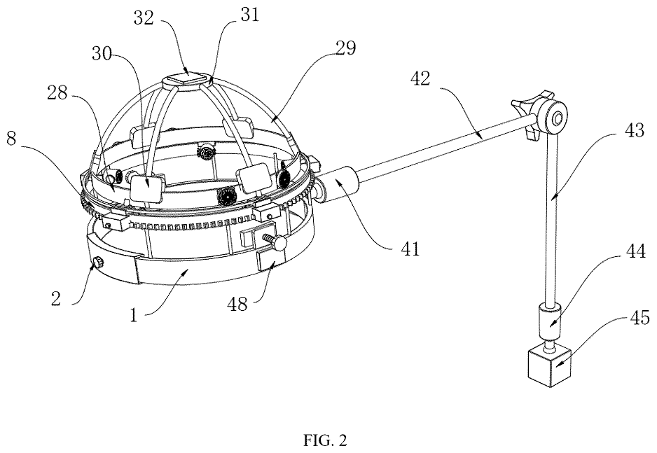

FIG. 3 is a schematic structural diagram of the head ring of the present disclosure.

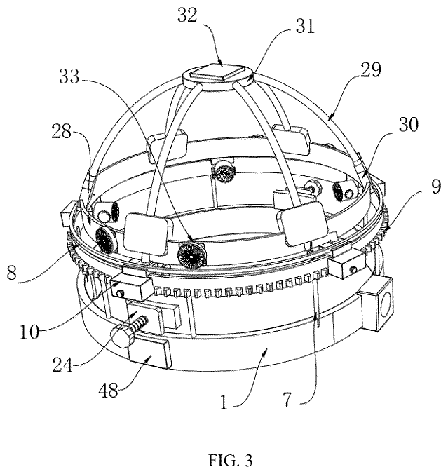

FIG. 4 is a schematic diagram of the internal structure of the protective shell of the present disclosure.

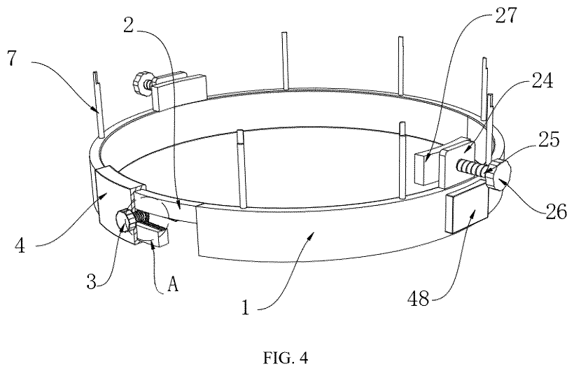

FIG. 5 is an enlarged view at A in FIG. 4.

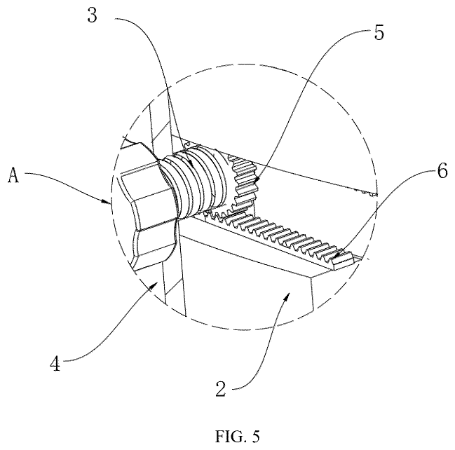

FIG. 6 is a schematic diagram of the internal structure of the toothed ring of the present disclosure.

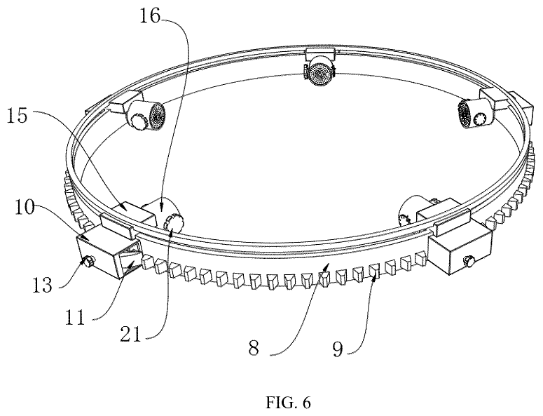

FIG. 7 is a schematic diagram of an internal structure of a moving block according to the present disclosure.

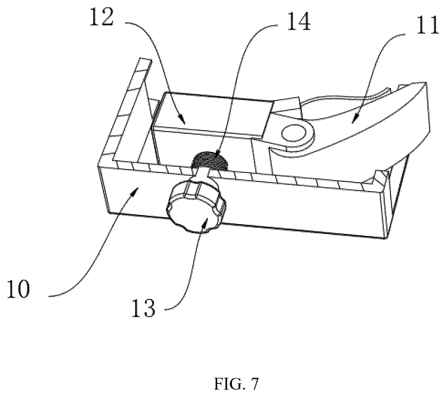

FIG. 8 is a schematic diagram of the internal structure of the ultrasonic generator of the present disclosure.

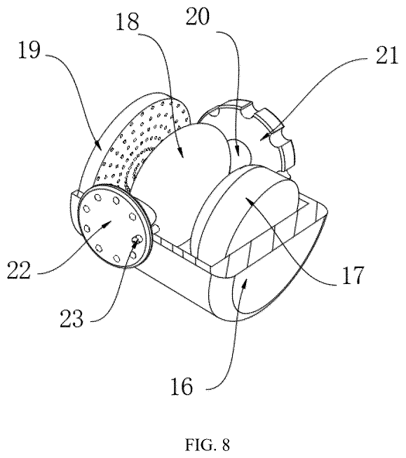

FIG. 9 is a schematic diagram of the internal structure of the housing of the present disclosure.

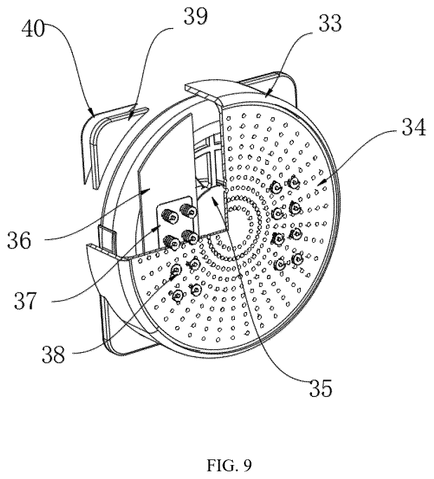

FIG. 10 is a structural schematic diagram of a part of the adjusting rod of the present disclosure.

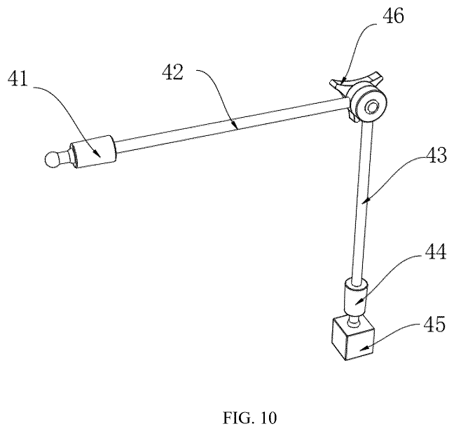

In the figures: 1, head ring; 2, connecting belt; 3, knob I; 4, protective shell; 5, gear; 6, rack; 7, connecting rod; 8, fixed ring; 9, toothed ring; 10, moving block I; 11, pawl; 12, connecting block; 13, knob II; 14, spring; 15, moving block II; 16, ultrasonic generator; 17, generator body; 18, acoustic lens; 19, dustproof net I; 20, rotating rod; 21, handle I; 22, turntable; 23, limit rod; 24, fixed plate; 25, screw; 26, handle II; 27, rubber pad; 28, water bag; 29, elastic band; 30, coil; 31, top plate; 32, biofeedback sensor; 33, shell; 34, dustproof net II; 35, fan; 36, mounting plate; 37, semiconductor refrigeration sheet; 38, radiating fin; 39, magnet I; 40, magnet II; 41, connector I; 42, adjusting rod I; 43, adjusting rod II; 44, connector II; 45, supporting block; 46, adjusting handle; 47, base; 48, temperature sensor; 49, controller; 50, indicator light; 51, display screen; 52, storage box; 53, processor; and 54, collector.

### DETAILED DESCRIPTION OF THE EMBODIMENTS

In the following, the technical scheme in the embodiment of the disclosure will be clearly and completely described with reference to the drawings in the specification of the disclosure. Obviously, the described embodiment is only a part of the embodiment of the disclosure, but not the whole embodiment. Based on the embodiments in the present disclosure, all other embodiments obtained by ordinary technicians in the field without creative labor belong to the scope of protection of the present disclosure.

### Embodiment

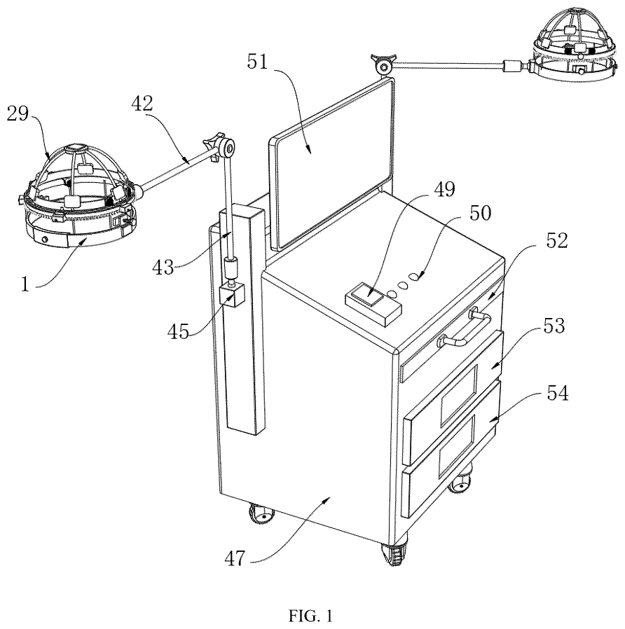

As shown in FIG. 1-F IG. 10, a closed-loop transcranial ultrasound brain stimulation device is provided in an embodiment of the present disclosure, including: a head ring 1, where the outer wall of the head ring 1 is fixedly connected with a vertically symmetrical connecting belt 2, the outer wall of the head ring 1 is fixedly connected with a protective shell 4, the inside of the protective shell 4 is rotatably connected with a knob I 3, the outer wall of the knob I 3 is fixedly connected with a gear 5, the lower surface of the connecting belt 2 is fixedly connected with a rack 6, and the rack 6 meshes with the gear 5; the upper surface of the head ring 1 is fixedly connected with a connecting rod 7, the upper surface of the connecting rod 7 is fixedly connected with a fixed ring 8, the outer wall of the fixed ring 8 is fixedly connected with a toothed ring 9, the outer wall of the fixed ring 8 is slidably connected with a moving block I 10, the inner part of the moving block I 10 is slidably connected with a knob II 13; the outer wall of the knob II 13 is fixedly connected with a connecting block 12, the inner part of the connecting block 12 is rotatably connected with a pawl 11; the pawl 11 is meshed with the toothed ring 9; the outer wall of the knob II 13 is sleeved with a spring 14, one end of the spring 14 is fixedly connected to the inside of the connecting block 12, and the other end of the spring 14 is fixedly connected to the inner wall of the moving block I 10; the outer wall of the moving block I 10 is fixedly connected to a moving block II 15, the outer wall of the moving block II 15 is slidably connected to the inside of the fixed ring 8, the outer wall of the moving block II 15 is fixedly connected to an ultrasonic generator 16, and the upper surface of the head ring 1 is provided with a fixed component.

Specifically, first, the knob I 3 is rotated, and the rotation of the knob I 3 drives the gear 5 to rotate inside the protective shell 4, thereby adjusting the size of the closed loop of the head ring 1. After the position and size of the head ring 1 are adjusted, the handle II 26 is rotated with the screw 25 inside the fixed plate 24, this process can make the rubber pad 27 closely fit with the patient's head, further ensuring that the head ring 1 closely fits with the patient's head. During the treatment, if the position of the ultrasonic sensor needs to be moved, the first moving block 10 can be pulled, and the movement of the moving block I 10 drives the pawl 11 to move on the outer wall of the fixed ring 8 under the action of the toothed ring 9, so as to adjust the specific position of the ultrasonic sensor according to the specific situation of the patient.

The fixed component includes a fixed plate 24, the lower surface of the fixed plate 24 is fixedly connected to the upper surface of the head ring 1, and a screw 25 is screwed in the fixed plate 24; one end of the screw 25 is fixedly connected with a handle II 26; the other end of the screw 25 is fixedly connected with a rubber pad 27; the inside of the ultrasonic generator 16 is fixedly connected with a generator body 17; the inside of the ultrasonic generator 16 is fixedly connected with a dustproof net I 19; the inside of the ultrasonic generator 16 is rotatably connected with a rotating rod 20, and the inside of the rotating rod 20 is fixedly connected with an acoustic lens 18, the outer wall of the acoustic lens 18 is arranged inside the ultrasonic generator 16; one end of the rotating rod 20 is fixedly connected with a handle I 21; the other end of the rotating rod 20 is fixedly connected with a turntable 22; a limit rod 23 is slidably connected inside the turntable 22; and the outer wall of the limit rod 23 is slidably connected inside the ultrasonic generator 16.

Specifically, if it is necessary to change the ultrasonic characteristics reflected by the ultrasonic sensor, the handle I 21 can be rotated with the rotating rod 20, and the rotating rod 20 drives the acoustic lens 18 to rotate inside the ultrasonic sensor, when the acoustic lens 18 moves to a proper position, the limit rod 23 is inserted into the ultrasonic sensor through the turntable 22 to fix the position of the acoustic lens 18, so that the focusing position of the ultrasonic can be changed as required. In the process of ultrasonic stimulation treatment, electromagnetic waves can also be generated by the coil 30 connected with the elastic band 29 to perform magnetic stimulation treatment on the patient. This design provides a variety of treatment options for the patient.

The outer wall of the head ring 1 is fixedly connected with a temperature sensor 48, the upper surface of the fixed ring 8 is fixedly connected with a centrally symmetric elastic band 29, the outer wall of the elastic band 29 is fixedly connected with a coil 30, the bottom end of the elastic band 29 is fixedly connected with a top plate 31, the upper surface of the top plate 31 is fixedly connected with a biofeedback sensor 32, the lower end of the outer wall of the elastic band 29 is fixedly connected with a water bag 28; the outer wall of the water bag 28 is fixedly connected with a magnet II 40, the outer wall of the magnet II 40 is provided with a magnet I 39, the outer wall of the magnet I 39 is fixedly connected with a shell 33, the inner wall of the shell 33 is fixedly connected with a dustproof net II 34, the inside of the shell 33 is fixedly connected with a fan 35, the inside of the shell 33 is fixedly connected with a mounting plate 36, and the inside of the mounting plate 36 is fixedly connected with a semiconductor refrigeration sheet 37, and the refrigeration surface of the semiconductor refrigeration sheet 37 is attached to the outer wall of the fan 35, and the heating surface of the semiconductor refrigeration sheet 37 is fixedly connected with a radiating fin 38, and one end of the radiating fin 38 is arranged on the outer wall of the shell 33; the inside of the head ring 1 is rotatably connected with a connector I 41, the outer wall of the connector I 41 is fixedly connected with an adjusting rod I 42, the inside of the adjusting rod I 42 is rotatably connected with an adjusting handle 46, and the outer wall of the adjusting rod I 42 is rotatably connected with an adjusting rod II 43, the bottom end of the adjusting rod II 43 is fixedly connected with a connector II 44, the outer wall of the connector II 44 is rotatably connected with a supporting block 45, and the outer wall of the supporting block 45 is fixedly connected with a base 47.

Specifically, the patient needs to sit in a special chair. The patient needs to sit in a special seat. When the patient is seated, pull the adjusting rod II 43 and rotate the connector II 44 on the supporting block 45. This step is to adjust the device to a proper angle for the patient's head. Next, the adjusting rod I 42 is rotated to drive the connector I 41 to rotate inside the head ring 1. The rotation of the adjusting lever drives the head ring 1 to move to the top of the patient's head, so as to make it closely fit with the top of the patient's head. This is to ensure the tightness between the head ring 1 and the patient's head. In the course of treatment, if it is necessary to cool the patient's head, firstly, the water bag 28 connected with the elastic band 29 is used to cool the patient's head. The cooling water in the water bag 28 can effectively prevent the brain from overheating. When the temperature of the water bag 28 or the patient's head rises, the temperature sensor 48 connected with the head ring 1 transmits information to the controller 49, and the controller 49 controls the indicator light 50 to light up to remind the doctor; if it is necessary to rapidly cool the patient's head, the shell 33 is connected with the magnet I 39 on the outer wall of the shell 33 with the magnet II 40. After the connection, the fan 35 and the semiconductor refrigeration sheet 37 in the shell 33 are started, and the heat generated by the heating surface of the semiconductor refrigeration sheet 37 is discharged from the shell 33 through the radiating fin 38. At the same time, the fan 35 blows out the cooled air to rapidly cool the cooling water in the water bag 28. This design greatly improves the user experience of the patient.

The inner upper end of the base 47 is connected with a storage box 52 in a sliding way, the upper surface of the base 47 is fixedly connected with a display screen 51, the upper surface of the base 47 is fixedly connected with a controller 49, the controller 49 is electrically connected with an indicator light 50, the outer wall of the indicator light 50 is fixedly connected with the upper surface of the base 47, the controller 49 is electrically connected with the temperature sensor 48; the inside of the base 47 is fixedly connected with a processor 53, a collector 54 is arranged below the processor 53, the outer wall of the collector 54 is fixedly connected with the inside of the base 47, and the collector 54 and the processor 53 are electrically connected.

Specifically, after close fitting, the patient's brain waves collected by the biofeedback sensor 32 on the top plate 31 of the elastic band 29 are collected by the collector 54 connected to the base 47, and then processed by the processor 53, and the processed data are displayed on the display screen 51 for doctors.

Although an embodiment of the present disclosure has been shown and described, it will be understood by those skilled in the art that various changes, modifications, substitutions and variations can be made to these embodiments without departing from the principles and spirit of the present disclosure, the scope of which is defined by the appended claims and their equivalents.

What is claimed is:

1. A closed-loop transcranial ultrasound brain stimulation device, comprising a head ring (1), wherein the outer wall of the head ring (1) is fixedly connected with a vertically symmetrical connecting belt (2), the outer wall of the head ring (1) is fixedly connected with a protective shell (4), the inside of the protective shell (4) is rotatably connected with a knob I (3), the outer wall of the knob I (3) is fixedly connected with a gear (5), the lower surface of the connecting belt (2) is fixedly connected with a rack (6), and the rack (6) meshes with the gear (5); the upper surface of the head ring (1) is fixedly connected with a connecting rod (7), the upper surface of the connecting rod (7) is fixedly connected with a fixed ring (8), the outer wall of the fixed ring (8) is fixedly connected with a toothed ring (9), the outer wall of the fixed ring (8) is slidably connected with a moving block I (10), the inner part of the moving block I (10) is slidably connected with a knob II (13); the outer wall of the knob II (13) is fixedly connected with a connecting block (12), the inner part of the connecting block (12) is rotatably connected with a pawl (11); the pawl (11) is meshed with the toothed ring (9); the outer wall of the knob II (13) is sleeved with a spring (14), one end of the spring (14) is fixedly connected to the inside of the connecting block (12), and the other end of the spring (14) is fixedly connected to the inner wall of the moving block I (10); the outer wall of the moving block I (10) is fixedly connected to a moving block II (15), the outer wall of the moving block II (15) is slidably connected to the inside of the fixed ring (8), the outer wall of the moving block II (15) is fixedly connected to an ultrasonic generator (16), and the upper surface of the head ring (1) is provided with a fixed component.

2. The closed-loop transcranial ultrasound brain stimulation device according to claim 1, wherein the fixed component comprises a fixed plate (24), the lower surface of the fixed plate (24) is fixedly connected to the upper surface of the head ring (1), and a screw (25) is screwed in the fixed plate (24); one end of the screw (25) is fixedly connected with a handle II (26); the other end of the screw (25) is fixedly connected with a rubber pad (27).

3. The closed-loop transcranial ultrasound brain stimulation device according to claim 1, wherein the inside of the ultrasonic generator (16) is fixedly connected with a generator body (17); the inside of the ultrasonic generator (16) is fixedly connected with a dustproof net I (19);
the inside of the ultrasonic generator (16) is rotatably connected with a rotating rod (20), and the inside of the rotating rod (20) is fixedly connected with an acoustic lens (18), the outer wall of the acoustic lens (18) is arranged inside the ultrasonic generator (16).

4. The closed-loop transcranial ultrasound brain stimulation device according to claim 3, wherein one end of the rotating rod (20) is fixedly connected with a handle I (21); the other end of the rotating rod (20) is fixedly connected with a turntable (22); a limit rod (23) is slidably connected inside the turntable (22); and the outer wall of the limit rod (23) is slidably connected inside the ultrasonic generator (16).

5. The closed-loop transcranial ultrasound brain stimulation device according to claim 1, wherein the outer wall of the head ring (1) is fixedly connected with a temperature sensor (48), the upper surface of the fixed ring (8) is fixedly connected with a centrally symmetric elastic band (29), the outer wall of the elastic band (29) is fixedly connected with a coil (30), the bottom end of the elastic band (29) is fixedly connected with a top plate (31), the upper surface of the top plate (31) is fixedly connected with a biofeedback sensor (32), the lower end of the outer wall of the elastic band (29) is fixedly connected with a water bag (28).

6. The closed-loop transcranial ultrasound brain stimulation device according to claim 5, wherein the outer wall of the water bag (28) is fixedly connected with a magnet II (40), the outer wall of the magnet II (40) is provided with a magnet I (39), the outer wall of the magnet I (39) is fixedly connected with a shell (33), the inner wall of the shell (33) is fixedly connected with a dustproof net II (34), the inside of the shell (33) is fixedly connected with a fan (35), the inside of the shell (33) is fixedly connected with a mounting plate (36), and the inside of the mounting plate (36) is fixedly connected with a semiconductor refrigeration sheet (37), and the refrigeration surface of the semiconductor refrigeration sheet (37) is attached to the outer wall of the fan (35), and the heating surface of the semiconductor refrigeration sheet (37) is fixedly connected with a radiating fin (38), and one end of the radiating fin (38) is arranged on the outer wall of the shell (33).

7. The closed-loop transcranial ultrasound brain stimulation device according to claim 1, wherein the inside of the head ring (1) is rotatably connected with a connector I (41), the outer wall of the connector I (41) is fixedly connected with an adjusting rod I (42), the inside of the adjusting rod I (42) is rotatably connected with an adjusting handle (46), and the outer wall of the adjusting rod I (42) is rotatably connected with an adjusting rod II (43).

8. The closed-loop transcranial ultrasound brain stimulation device according to claim 7, wherein the bottom end of the adjusting rod II (43) is fixedly connected with a connector II (44), the outer wall of the connector II (44) is rotatably connected with a supporting block (45), and the outer wall of the supporting block (45) is fixedly connected with a base (47).

9. The closed-loop transcranial ultrasound brain stimulation device according to claim 8, wherein the inner upper end of the base (47) is connected with a storage box (52) in a sliding way, the upper surface of the base (47) is fixedly connected with a display screen (51), the upper surface of the base (47) is fixedly connected with a controller (49), the controller (49) is electrically connected with an indicator light (50), the outer wall of the indicator light (50) is fixedly connected with the upper surface of the base (47), the controller (49) is electrically connected with the temperature sensor (48).

10. The closed-loop transcranial ultrasound brain stimulation device according to claim 8, wherein the inside of the base (47) is fixedly connected with a processor (53), a collector (54) is arranged below the processor (53), the outer wall of the collector (54) is fixedly connected with the inside of the base (47), and the collector (54) and the processor (53) are electrically connected.
